(19) United States
(12) Plant Patent Application Publication
(10) Pub. No.: US 2001/0013136 P1
(43) Pub. Date: Aug. 9, 2001

(54) Chrysanthemum named White Reagan Mundo

(76) Inventor: Robert Noodelijk, Woubrugge (NL)

Correspondence Address: PARKHURST & WENDEL, 1421 PRINCE STREET, SUITE 210, ALEXANDRIA, VA, 22314, US

(21) Appl. No.: 09/773,608

(22) Filed: Feb. 2, 2001

(30) Foreign Application Priority Data
Jul. 15, 1998 (EP) 1998/0974
Jul. 21, 1997 (NL) CHR 2945
Mar. 18, 1999 (PL) O1047

(51) Int. Cl. A01H 5/00

(52) U.S. Cl. PLT/294

(57) ABSTRACT

A Chrysanthemum plant named WHITE REAGAN MUNDO characterized by its medium sized blooms with white ray-florets and yellow-green disc florets.

### RELATED APPLICATIONS

The present application is related to the following co-pending applications: Application no. Filing Date Title

### BACKGROUND OF THE INVENTION

The new plant of the present invention comprises a new and distinct cultivar of chrysanthemum plant that is a natural occuring sport of a parent chrysanthemum named PEARL REAGAN MUNDO. A comparison with Parent chrysanthemum PEARL REAGAN MUNDO is also made in this application. The new cultivar was discovered as a whole plant mutation in September 1996 by Rob Noodelijk in a controlled environment (greenhouse) in Rijsenhout Holland. The first act of asexual reproduction of WHITE REAGAN MUNDO was accomplished when vegetative cuttings were taken from the initial selection in November 1996 in Rijsenhout Holland.

### SUMMARY OF THE INVENTION

The present invention is a new and distinct variety of chrysanthemum bearing medium sized blooms with white ray-florets and yellow-green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a stem of the cultivar in full bloom.

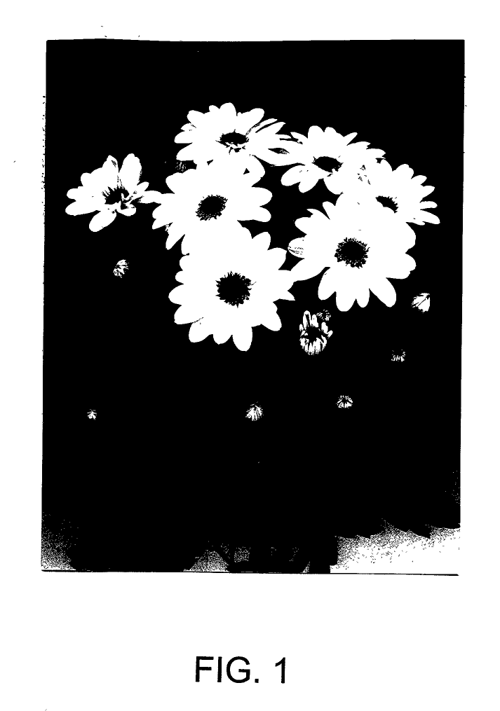

### DESCRIPTION OF THE INVENTION

This new variety of chrysanthemum is of the botanical classification Dendranthema grandiflora. The observations and measurements were gathered from plants grown in a greenhouse in Rijsenhout Holland in a photo-periodic controlled crop. The photo-periodic response time in this crop was 50 days. This new variety produces medium sized blooms with white ray-florets and yellow-green disc-florets blooming on the plant for 28 days. This new variety of chrysanthemum has been found to retain its distinctive characteristics throughout successive propagations however the phenotype may vary significantly with variations in environment such as light intensity and temperature. To show the phenotype as described WHITE REAGAN MUNDO may be planted without assimilation lightning between week 50 and 40 under greenhouse conditions in Holland. With assimilation lightning (minimum level 2500 lux) it may be planted year round under greenhouse conditions in Holland.

The variety WHITE REAGAN MUNDO is a natural mutant of PEARL REAGAN MUNDO. The variety PEARL REAGAN MUNDO is a natural mutant of REAGAN. When WHITE REAGAN MUNDO, PEARL REAGAN MUNDO and REAGAN are being compared the following differences and similarities are noticed: (A) The difference of WHITE REAGAN MUNDO and PEARL REAGAN MUNDO is the flower color for which characteristic WHITE REAGAN MUNDO has been selected out of PEARL REAGAN MUNDO. All other characteristics of WHITE REAGAN MUNDO and PEARL REAGAN MUNDO are the same. (B) The differences of PEARL REAGAN MUNDO and REAGAN are (1) flower form (2) flower size (3) flower color (4) spray formation and flower count and (5) tolerance for a lower night temperature for which characteristics PEARL REAGAN MUNDO has been selected out of REAGAN. All other characteristics of PEARL REAGAN MUNDO and REAGAN are similar. (A) Flower color: WHITE REAGAN MUNDO has white ray-florets instead of pale pink ray-florets. (B) (1) Flower form: The ray-florets of PEARL REAGAN MUNDO are less broad and more pointed and they are more upright. The flower-disc is smaller, greener and more regular. (2) Flower size: The flower is a little smaller. (3) Flower color: The flower color is paler (4) The spray formation: The spray formation is more flat, less pyramidal, mostly a little wider and the flower count is higher. (5) Temperature tolerance: PEARL REAGAN MUNDO and all its color mutants can be grown at a lower night temperature. The required minimum temperature for REAGAN (under all circumstances) is 17° C. Under high light conditions PEARL REAGAN MUNDO can be grown at 14° C. This has been tested and confirmed in poor heated crops under high light conditions flowering in February 1997 and 1998 in Italy and Spain.

The following is a description of the plant and characteristics that distinguish WHITE REAGAN MUNDO as a new and distinct variety.

The color designations are taken from the plant itself. Accordingly, any discrepancies between the color designations and the colors depicted in the photographs are due to photographic tolerances.

Botanical Description of

| | CULTIVAR WHITE REAGAN |
|---|---|
| Bud | |
| Size | Medium Cross-section +/−1.0 |
| | cm height +/−0.7 cm |
| Outside Color | Yellow 4 D |
| Bloom | |
| Size | Medium |
| Fully Expanded | 6-6.5 cm |
| Number of blooms per single stem | Average of 12 |
| Peduncle length | Near the top +/−11 cm, |
| | near the middle +/−18 cm, |
| | near the bottom +/−23 cm |
| Peduncle lateral shoot attachment | Medium |
| Peduncle lateral shoot angle | Medium to large, 40° |
| Form | Daisy |
| Performance (blooming period) | 28 days |
| Color | |
| Center of the flower (disc-florets) | Immature Yellow-green 144 A |
| | mature Yellow-green 144 B |
| Color of upper surface of the | White 155 D |
| majority of the ray-florets | |
| Color of the lower surface of the | White 155 D |
| majority of the ray-florets | |
| Tonality from Distance | A white daisy with a yellow- |
| | green disc |
| Discoloration | None |
| Ray florets | |
| Texture | Upper and under side smooth |
| Number | 26-30 (two rows) |
| Cross-section | Convex |
| Longitudinal axis of majority | Straight to slightly reflexing |
| Shape of tip | Pointed |
| Fragrance | Typical chrysanthemum |
| Disc Diameter (when visible) | Medium (1.3-1.5 cm) |

| | PEARL | |
|---|---|---|
| | REAGAN MUNDO | REAGAN MUNDO |
| Bud | | |
| Size | Medium Cross- | |
| | section +/−1.0 cm | |
| | height +/−0.7 cm | |
| Outside Color | Yellow-white 158 D | Greyed-purple |
| | | 186 D |
| Bloom | | |
| Size | Medium | |
| Fully Expanded | 6-6.5 cm | 6.5-7 cm |
| Number of blooms per | Average of 12 | 10 |
| single stem | | |
| Peduncle length | Near the top +/−11 cm, | Near the top +/−14 |
| | near the middle +/−18 | cm, near The |
| | cm, near the bottom | middle +/−24 cm, |
| | +/−23 cm | near The bottom |
| | | +/−28 cm |
| Peduncle lateral shoot | Medium | |
| attachment | | |
| Peduncle lateral shoot | Medium to large, 40° | Medium, 30° |
| angle | | |
| Form | Daisy | |
| Performance | 28 days | |
| (blooming period) | | |
| Color | | |
| Center of the flower | Immature Yellow-green | Immature Yellow- |
| (disc-florets) | 144 B | green 151 C |
| | Mature Yellow- | Mature Yellow- |
| | green 154 A | green 154 A |
| Color of upper surface of | Red-purple 69 B but | Red-purple 75 B |
| the majority of the | paler | |
| ray-florets | | |
| Color of the lower | Red-purple 75 D but | Red-purple 69 D but |
| surface of the majority of | paler | paler |
| the ray-florets | | |
| Tonality from Distance | A pearl pink daisy with | |
| | a yellow-green disc | |
| Discoloration | None | |
| Ray florets | | |
| Texture | Upper and under side | |
| | smooth | |
| Number | 26-30 (two rows) | 24-30 (two rows) |
| Cross-section | Convex | |
| Longitudinal axis of | Straight to slightly | Reflexing |
| majority | reflexing | |
| Shape of tip | Pointed | Dentate |
| | | occasionally pointed |
| Fragrance | | |
| Disc Diameter (when | Medium (1.3-1.5 cm) | Medium (1.5 cm) |
| visible) | | |

Reproductive Organs

| | | |
|---|---|---|
| Stamen (present in | Yellow, thick, 3 mm in | Yellow, thick, 3 mm in |
| disc florets only) | length | length |
| Pollen | Present | Present |
| Styles (present in | Green, thick | Green, thick |
| both ray and disc | | |
| florets) | | |
| Style Length | +/−5 mm | +/−5 mm |
| Stigmas | Yellow | Yellow |
| Stigma Width | +/−1 mm | +/−1 mm |
| Ovaries | Enclosed in calyx | Enclosed in calyx |
| Plant | | |
| Form | A spray mum meant for | A spray mum meant for |
| | erect culture | erect culture |
| Growth | Medium | Medium |
| Height | 100-115 cm | 100-115 cm |
| Stem Color | Yellow-green 146 B | Yellow-green 146 B |
| Stem Strength | Medium to strong | Medium to strong |
| Stem Brittleness | Absent | Absent |
| Stem Anthocyanin | Present | Present |
| Coloration | | |
| Flowering Response | 50 days | 50 days |
| (photo-periodic | | |
| controlled crop, no | | |
| natural growing) | | |
| Foliage | | |
| Color | Upper side green 137 A | Upper side green 137 A |
| | under side green 137 C | under side green 137 C |
| Size | length +/−12 cm, | length +/−12 cm, |
| | width +/ 9 cm | width +/ 9 cm |
| Quantity (number | 26-32 | 26-32 |
| per single stem) | | |
| Shape | Lobed | Lobed |
| Texture | Fleshy | Fleshy |
| Ribs and Veins | Ribs and veins well | Ribs and veins well |
| | developed | developed |
| Edge | Crenated | Crenated |
| Shape of Base of | Round | Round |
| Sinus Between | | |
| Lateral Lobes | | |
| Margin of Sinus | Converging sometimes | Converging sometimes |
| Between Lateral | asymmetric | asymmetric |
| Lobes | | |
| Shape of Base | Rounded | Rounded |
| Apex | Mucronate | Mucronate |

I claim:

1. A new and distinct variety of chrysanthemum plant as described and illustrated.
